(19) United States
(12) Patent Application Publication
(10) Pub. No.: US 2003/0153800 A1
(43) Pub. Date: Aug. 14, 2003

(54) Use of quasi-crystalline aluminum alloys in applications in refining and petrochemistry

(75) Inventors: Philippe Lecour, Les Mureaux (FR); Xavier Longaygue, Noisy Le Roi (FR); Francois Ropital, Rueil Malmaison (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: Institut Francais du Petrole, Rueil Malmaison Cedex (FR)

(21) Appl. No.: 10/307,517

(22) Filed: Dec. 2, 2002

(30) Foreign Application Priority Data
Nov. 30, 2001 (FR) 01/15.480

(51) Int. Cl. C07C 5/327; C07C 5/333; C10G 9/36; C10G 35/00; C10G 45/00

(52) U.S. Cl. 585/654; 208/133; 208/130; 208/208 R

(57) ABSTRACT

Materials that consist at least in part of aluminum quasi-crystals whose composition is represented by the general formula:

AlₐCu_{b}Co_{c}FE_{d}CrₑM_{f}I_{g},

in which M represents one or more additional minor elements and I represents one or more alloy impurities and with, in terms of percentage of atoms, 0<b<30, 0<c<30, 0<d<20, 0<e<20, 0<f<10, 0<g<2, and a+b+c+d+e+f+g=100, are used in the manufacture of devices or device parts, for example, tubes, plates, or hoops, for building furnaces, reactors, or pipes, or for lining the inner walls of reactors, furnaces, or pipes inside of which conditions can prevail for coke formation, carburization, sulfurization, nitration, oxidation, or attack by halogenating agents when refining and petrochemical processes are implemented.

### BACKGROUND OF THE INVENTION

1. Field of the Invention

This invention pertains to the use of aluminum-based quasi-crystalline alloys in applications in refining and petrochemistry.

According to the invention, these alloys can be used in particular in the fabrication of parts, for example, tubes, plates, or hoops for building reactors, furnaces, or pipes, or for lining the inner walls of furnaces, reactors or pipes, inside of which conditions can prevail for coke formation, carburization, sulfurization, nitration, oxidation, or attack by halogenating agents when refining and petrochemical processes are implemented that take place at temperatures of between, for example, 350° C. and 1100° C.

The invention also pertains to reactors, furnaces, and pipes or parts thereof that are built or lined with these alloys.

The carbon deposit that develops in furnaces and reactors during hydrocarbon conversion is generally referred to as coke. This coke deposit has an adverse effect in industrial units. In point of fact, coke formation on the walls of tubes and reactors causes, in particular, a reduction in heat exchange, considerable blockage, and thus an increase in losses of feedstock. In order to maintain a constant reaction temperature, it may be necessary to raise the temperature of the walls, and this carries with it the risk of damaging the alloy of which these walls are made. A reduction in the selectivity of the systems and thus in yield is also observed. Moreover, these coke deposits can cause carburization of materials made of metal.

When elevated contents of sulfurous products are present in the feedstocks, significant losses of the thickness of the walls of reactors and their internal, as well as in furnaces, develop starting at 300° C. for the majority of the alloys that are currently in use. In order to correct this problem, these feedstocks cannot be used at present with such contents of sulfurous products, and separation has to be done at temperatures of less than 300° C. Likewise, for certain ammonia cracking processes, the reactors, furnaces, and equipment need to be resistant to sulfurization and nitration at temperatures of between 300 and 1100° C. Today, only the use or ceramic materials can meet these requirements as regards chemical resistance at high temperatures; owing, however, to their high mechanical fragility, especially during rapid changes in temperature, it is almost impossible to utilize these materials on an industrial basis.

In the processes that operate with in-situ regeneration of the catalyst, injecting a halogenating agent (based on chlorine, for example) at between 300 and 800° C. can cause corrosion and thus a significant loss of thickness in the regeneration reactors, particularly above 600° C. The behavior of the alloys that are used today limits the concentration of the regeneration agent and the regeneration temperature and thus makes it impossible to optimize this catalyst regeneration process.

2. Description of the Prior Art

European Patents Nos. 0 356 287 and 0 521 138, which describe lining materials for metallic alloys and metals, are known.

European Patent No. 0 5040 48, which describe the creation of strands by thermal projection for the purpose of depositing a quasi-crystalline phase, is also known.

Moreover, U.S. Patent Application 2001/0,001,967 A and European Patent No. 0 587 186 describe aluminum alloys with enhanced mechanical properties. In the second document, the presence of intermetallic phases or such as Ni₃Al mainly accounts for these properties.

U.S. Pat. Nos. 6,242,108 B,-6,254,699 B, and 6,254,700 B, which describe quasi-crystalline alloy linings that are resistant to wear, tear, and abrasion, are known.

### SUMMARY OF THE INVENTION

For its part, this invention pertains to the use of a material that consists at least partially and preferably largely of a quasi-crystalline aluminum alloy with a composition that is designed to ensure good resistance to coking, carburization, sulfurization, nitration, oxidation, or attack by halogenating agents.

### BRIEF DESCRIPTION OF THE DRAWING

FIG. 1 shows the weight gain curves caused by coking for different steels and alloys considered in the invention.

### DETAILED DESCRIPTION OF THE INVENTION

The compositions of the quasi-crystalline aluminum alloys that are contemplated by this invention can be represented by the general formula AlₐCu_{b}Co_{c}Fe_{d}CrₑM_{f}I_{g}, in which M represents one or more additional minor elements and I represents one or more alloy impurities and with, in terms of percentage of atoms, 0<b<30; 0<c<30; 0<d<20; 0<c<20; 0f<10; 0<g<2; and a+b+c+d+e+f+g=100.

The additional minor elements M can be selected from among, for example: B, C, Mn, Ni, W, Nb, Ti, Si, Mo, Mg, Zn, V, Y, Ru, Os, Pd, Zr, Rh, Ta, and Y.

Impurities I are virtually inevitable and come from the working of the alloy. They can consist of, for example (whereby this list is not exhaustive), N, O, Ca, S, Sn, As, P and/or Sb.

In other words, the compositions of the alloys of the general formula:

AlₐCu_{b}Co_{c}Fe_{d}CrₑM_{f}I_{g}

contain, in terms of atoms, 0 to 30% copper, 0 to 30% cobalt, 0 to 20% iron, 0 to 10% chromium, 0 to 10% of at least one element M, and 0 to 2% of alloy impurities, whereby the rest of the composition up to 100% consists of aluminum.

According to the invention, the alloys as defined above can be used for the manufacture of parts or devices such as tubes, plates, or hoops that are designed to be used to build furnaces, reactors, or pipes, whereby these parts or devices generally consist of multiple pieces.

It is also possible to utilize the alloy according to the invention to line the inside walls of furnaces, reactors, or pipes by at least one of the following techniques: co-centrifuging, thermal projection (plasma, electric arc, flame process), PVD (physical vapor deposition), CVD (chemical vapor deposition), electrolysis, the sol-gel technique, electrophoresis, laser, “overlay,” or plating.

The alloy that is used in this invention can be worked by classical foundry or casting techniques and then shaped by the techniques that are commonly used to fabricate the desired parts: plates, sheets, grids, tubes, sections, etc. These semi-finished parts can then be used to build the main parts of furnaces or reactors or simply as accessory or auxiliary parts for such devices.

The alloy according to this invention can be used in the form of a powder to make linings for the inside walls of reactors, grids, or tubes or to produce parts by compaction.

An alloy of this type can be used to manufacture facilities that implement petrochemical processes, for example, reforming, steam-cracking, vapor reforming, catalytic or thermal cracking, dehydrogenation, desulfurization, and catalyst regeneration. Such chemical reactions take place at temperatures of between 350° C. and 1100° C.

A first particular application is the catalytic reforming reaction, which makes it possible to obtain a reformate at between 450° C. and 650° C., during which the secondary reaction leads to coke formation.

Another particular application is the steam-cracking of naphtha at 800-1100° C.

Another particular application is the ammonia cracking process that is carried out between 300 and 800° C., which involves phenomena of sulfurization and nitration.

Another particular reaction is the isobutane dehydrogenation process, which makes it possible to obtain isobutene at between 550° C. and 700° C.

Another particular application is the desulfurization of refined products, which is carried out at temperatures of 300° C. to 800° C.

Another particular application is the in-situ regeneration of catalysts by halogenating agents, for example chlorinating agents (involving phenomena of oxy-halogenation, especially oxy-chlorination), which is carried out at temperatures of 300° C. to 750° C.

The invention will be better understood and its advantages made clearer by reading the following examples and tests, which are in no way limiting.

### EXAMPLES

The alloys used are two austenitic steels (steels A and B) for purposes of comparison, and two quasi-crystalline alloys (alloys C an D). Steels A and B are standard austenitic stainless steels that are currently used for building reactors or parts of reactors. Alloy C consists basically of a quasi-crystal Al₆₇Cu₁₈Fe₁₀Cr₅. Alloy D consists essentially of a quasi-crystal Al₇₁Cu₁₃Fe₆Cr₈. Table 1 below indicates the compositions by weight of these alloys.

TABLE 1
Compositions of the Alloys (% by Weight)

| Alloy | C | Mn | Ni | Cu | Cr | Fe | Co | Al |
|---|---|---|---|---|---|---|---|---|
| A | 0.1 | 0.6 | 20 | — | 25 | Base | — | — |
| B | 0.08 | 1.5 | 11 | — | 18 | Base | — | — |
| C | — | — | — | 30 | 7 | 15 | — | 48 |
| D | — | — | — | — | 11.7 | 12.7 | 21.6 | 54 |

Example 1:

A catalytic reforming test was carried out at 650° C.

The working protocol used to carry out the tests is as follows:

each alloy sample is sliced off by electro-erosion, and then is polished with SiC #180 paper to ensure a standard surface state and to remove the oxide film that can form during cutting;

degreasing is done in a CCl₄ bath with acetone and then ethanol; the sample is suspended on the arm of a thermobalance;

the tube reactor is closed, and the temperature is raised under argon.

The feedstock is a naphtha. It is a hydrocarbon mixture composed of paraffins, naphthenes, and aromatic compounds (with a P/N/A ratio of 61/29/10). The naphtha feedstock is injected in liquid form by a syringe driver, and the feedstock is then converted into gaseous form in the evaporator. A hydrocarbon flow rate that is equal to 13 ml/hour (flow rate of the liquid feedstock at 20° C., which corresponds to a gas flow rate of 31.9 ml/hour), and a hydrogen gas flow rate that is equal to) 190 ml/hour are selected. The H₂/hydrocarbon ratio is thus equal to 6.

The microbalance makes it possible to continuously measure the gain weight on the sample and to deduct therefrom a coking rate that is expressed as the gain weight per unit of time and per unit of surface of the sample.

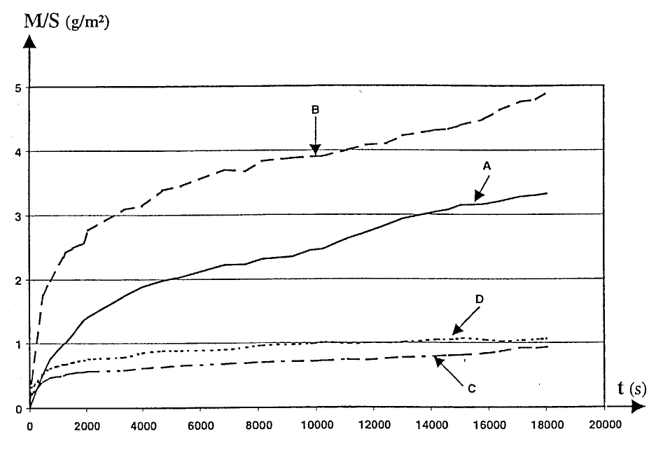

FIG. 1 shows the weight gain curves caused by coking for different steels and alloys under study. This figure demonstrates that the coking of the samples of standard steels A and B is significantly greater than that of the quasi-crystals C and D.

Table 2 below indicates the values of the asymptotic coking rate (g/hm²), as can be derived from the curves in FIG. 1.

TABLE 2

| Alloy | Asymptotic Coking Rate (g/hm²) |
|---|---|
| A | 0.30 |
| B | 0.60 |
| C | 0.11 |
| D | 0.02 |

Example 2:

A high-temperature oxidation test was carried out. This test was conducted at a temperature of 900° C. under air. The protocol for the preparation of the samples is the same as that presented in Example 1. Heating to 900° C. is done under argon. Once this temperature is reached, air is injected into the reactor. The weight gain of the sample is recorded as a function of time.

All of the oxidation curves obtained have a parabolic plot. As a matter of fact, an oxide layer can grow according to a parabolic pattern, which indicates that the growth of the layer is thus controlled by diffusion. The kinetics of this kind of reaction is written according to the following formula:

(ΔM/S)²=kₚt

with t=time in seconds;

ΔM/S=weight gain per unit of surface squared in gm⁻²;

kₚ=diffusion constant in g²·m⁻⁴·sec⁻¹.

Table 3 below gives the values of the diffusion constant kₚ (in g²·m⁻⁴·sec⁻¹) that were obtained for steel B and the two quasi-crystalline alloys C and D:

TABLE 3

| | Diffusion Constant |
|---|---|
| Alloy | kₚ (g² · m⁻⁴ · sec⁻¹) |
| B | 2 10⁻⁵ |
| C | 3.2 10⁻⁶ |
| D | 1.3 10⁻⁵ |

Example 3:

A high-temperature oxy-chlorination test was carried out. The tests are carried out at 600 and 650° C. A gaseous mixture composed of water, oxygen, nitrogen, and a chlorinating agent (dichloropropane) is used. The most stringent operating conditions are used, i.e., with an elevated molar percentage of chlorine.

The composition of the gaseous mixture is given in Table 4 below:

TABLE 4
Composition of the Gaseous Reaction Mixture

| | Molar Percentage | Molar Flow rate in | Volumetric Flow |
|---|---|---|---|
| | in the | the Feed, in | Rate in the Feed, in |
| Product | Reactor | mmol/hour | μl/hour |
| C₂Cl₄ | 0.075 | 0.75 | 770 |
| N₂ | 64 | 640 | 256 |
| O₂ | 16 | 160 | 64 |
| H₂O | 1 | 10 | 180 |
| Ar | 18.295 | 189.25 | 64 |

The protocol for the preparation of the samples is the same as that presented in Example 1. Heating to 600° C. or 650° C. is done under argon. Once this temperature is reached, the reaction mixture is injected into the reactor. From the curves of weight gain as a function of time, the rates of weight gain for all of the samples at 600° C. and 650° C. are calculated.

The results obtained are presented in Table 5 below:

TABLE 5

| Alloy | Rate in g · m⁻² · h⁻¹ at 600° C. | Rate in g · m⁻² · h⁻¹ at 650° C. |
|---|---|---|
| A | [blacked out] | 2.350 |
| B | 0.260 | 1.430 |
| C | 0.002 | 0.002 |
| D | 0.012 | 0.099 |

Example 4:

A test was carried out on sulfurization and nitration. This test is carried out at a temperature of 700° C. in a gaseous mixture with the following composition: 15% H₂O, 13% N₂, 42% H₂, and 30% H₂S. The protocol for the preparation of the samples is the same as presented in Example 1. The samples are weighed and then suspended in a quartz ampoule. The ampoule is placed in a furnace that is brought to the test temperature (700° C.). The gaseous mixture is kept circulating for several tens of hours. At the end of the tests, the samples are weighed again after the corrosion deposits are removed, and the rate of corrosion is calculated from the loss of weight.

Table 6 below gives the values for the rates of corrosion (mm/year) for the different steels and alloys that were tested:

TABLE 6

| Alloy | Rate of Corrosion (mm/year) |
|---|---|
| A | 24 |
| B | 34 |
| C | <0.1 |
| D | <0.1 |

The preceding examples can be repeated with similar success by substituting the generically or specifically described reactants and/or operating conditions of this invention for those used in the preceding examples.

The entire disclosures of all applications, patents and publications, cited herein and of corresponding French application No. 01/15.480, filed Nov. 30, 2001 are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. Use of a material that consists at least partially of a quasi-crystalline aluminum alloy whose composition is represented by the general formula:
AlₐCu_{b}Co_{c}Fe_{d}CrₑM_{f}I_{g}
in which M represents one or more additional minor elements and I represents one or more alloy impurities and with, in terms of percentage of atoms, 0<b<30; 0<c<30; 0<d<20; 0<e<20; 0<f<10; 0g<2; and a+b+c+d+e+f+g=100, in the manufacture or lining of a device or part of a device that has improved properties of resistance to coking, carburization, sulfurizatiton, nitration, oxidation, or halogenated agents:

2. Use according to claim 1, characterized in that in the composition of the alloy, the additional element M is selected from among B, C, Mn, Ni, W, Nb, Ti, Si, Mo, Mg, Zn, V, Y, Ru, Os, Pd, Zr, Rh, Ta, and Y.

3. Use according to claim 1 or 2, wherein in the composition of the alloy, the impurity I is selected from among N, O, Ca, S, Sn, As, P, and Sb.

4. Device or device part that has improved properties of resistance to coking, sulfurization, nitration, oxidation, or halogenated agents, wherein it is manufactured from at least one material as defined in one of claims 1 to 3.

5. Device or device part that has improved properties of resistance to coking, sulfurization, nitration, oxidation, or halogenated agents, wherein it is lined with a material as defined in one of claims 1 to 3.

6. Method of producing a device or device part according to claim 4, wherein said device or said device part is composed of multiple pieces.

7. Method of lining a device or device part according to claim 5, wherein at least one of the techniques selected from among co-centrifuging, thermal projection (plasma, electric arc, flame process), PVD (physical vapor deposition), CVD (chemical vapor deposition), electrolysis, the sol-gel technique, electrophoresis, laser, “overlay,” or plating is used.

8. Use of a device according to claim 4 or 5 or manufactured by a method according to claim 6 or lined by a method according to claim 7, in the implementation of a petrochemical process that takes place at temperatures of 350° C. to 1100° C.

9. Use according to claim 8, wherein said petrochemical process is a catalytic reforming process that makes it possible to obtain the reformate at temperatures of 450° C. to 650° C.

10. Use according to claim 8, wherein said petrochemical process is a naphtha steam-cracking process for temperatures of 800° C. to 1100° C.

11. Use according to claim 8, wherein said petrochemical process is an ammonia cracking process for temperatures of 300° C. to 800° C.

12. Use according to claim 8, wherein said petrochemical process is an isobutane dehydrogenation process that makes it possible to obtain isobutene at temperatures of 550° C. to 700° C.

13. Use according to claim 8, wherein said petrochemical process is a catalyst regeneration process that is carried out at temperatures of 300° C. to 750° C.

14. Use according to claim 8, wherein said petrochemical process is a process for desulfurization of refined products at temperatures of 300° C. to 800° C.
